(19) United States
(12) Patent Application Publication
(10) Pub. No.: US 2011/0054352 A1
(43) Pub. Date: Mar. 3, 2011

(54) Portable Dialysis Access Monitor Device

(76) Inventors: Po-Jen Ko, Taipei City (TW); Yun-Hen Liu, Guishan Shiang (TW); Ching-Yang Wu, Linkou Township (TW)

(21) Appl. No.: 12/546,739

(22) Filed: Aug. 25, 2009

(51) Int. Cl. A61B 7/00

(52) U.S. Cl. 600/586

(57) ABSTRACT

A portable dialysis access monitor device, comprising: an electronic monitor device and a server. The electronic monitor device includes a first microprocessor, an acoustic detector, a touch screen, a first storage device, a diagnostic programming module, an alarm device and a power supply device. The first microprocessor collects acoustic frequency signals, sound signals and thrill signals detected from a dialytic fistula. If signal difference is greater than a predetermined threshold value, the electronic monitor device provides a warning by the alarm device. The server includes a second microprocessor, a second storage device and a mobile internet. The server receives and analyzes the acoustic frequency signals, the sound signals and the thrill signals detected from the dialytic fistula by utilizing the second microprocessor to get an analyzed result. The server transmits the analyzed result to the electronic monitor device by utilizing the mobile internet.

### BACKGROUND OF THE INVENTION

1. Field of the Invention

The present invention relates to a monitor device, and more particularly to a portable dialysis access monitor device.

2. Description of Related Art

With respect to patients with end-stage renal disease, to have a reliable dialytic fistula is crucial. They need to be removed metabolic waste products or toxic substances from the bloodstream by utilizing a dialytic fistula and a dialysis equipment in order to maintain a constant physical environment. As to patients who have to receive a long-term hemodialysis access, how to keep away from blood clots clogged in the dialytic fistula is an important issue. Even in Nowadays, how to effectively clear the blood clots clogged in the dialytic fistula and rectify the cause resulting in the formation of the blood clots at the same time is still a big challenge for vascular surgeons.

With the development of invasive treatment technology, transluminal arterioplasty and embolectomy have already made certain progress. However, to prevent the the formation of the blood clots in the dialytic fistula at initial stage is still dependent heavily on vascular surgeons by their clinical experience and their subjective physical examination such as attenuated vascular thrill or eliminated vascular thrill. It is often occurred that the dialytic fistula of the dialysis patient can't function properly to carry out hemodialysis. Then, the dialysis patient has to be transferred to receive an invasive surgical treatment in order to restore the dialytic fistula. However, in recent clinical experience, it is often occurred that the dialytic fistula is in poor internal condition so that it needs to implant a Hickmen catheter or a double lumen catheter urgently. It not only results in the waste of medical resources but also making the dialysis patient suffer a lot mentally and physically. Nevertheless, conventional dialytic fistula is lack of an effective monitor device to monitor the blood flow therein. Therefore, the possible problems of the dialytic fistula can't be diagnosed early and effectively. Early diagnosis of the vascular blockage is needed in order to minimize risk to the dialysis patient and to maximize likelihood that treatment will successfully reduce the blockage to a safe level or eliminate the blockage completely. Thus, the need for improvement still exists.

### SUMMARY OF THE INVENTION

The main objective of the present invention is to provide a portable dialysis access monitor device, comprising: an electronic monitor device and a server. The electronic monitor device includes a first microprocessor, an acoustic detector, a touch screen, a first storage device, a diagnostic programming module, an alarm device, a power supply device and an interface adopted to be between a human and a machine. The first microprocessor is electrically connected to the touch screen, the first storage device, the alarm device and the power supply device respectively. The interface is a graphical user interface. The first microprocessor collects acoustic frequency signals, sound signals and thrill (e.g., a vascular murmur) signals detected from a dialytic fistula and stored in the first storage device. The first microprocessor utilizes the diagnostic programming module to diagnose. If signal difference is greater than a predetermined threshold value, the electronic monitor device provides a warning by the alarm device.

Besides, the server includes a second microprocessor, a second storage device and a mobile internet. The server is connected to the electronic monitor device by utilizing the mobile internet; wherein the server receives and analyzes the acoustic frequency signals, the sound signals and the thrill signals detected from the dialytic fistula by utilizing the second microprocessor to get an analyzed result. The server transmits the analyzed result to the electronic monitor device by utilizing the mobile internet and the analyzed result is stored in the second storage device.

By utilizing the invention, the following advantages are obtained. First, the present invention provides a portable dialysis access monitor device for detecting possible problems of the dialytic fistula and utilizes the diagnostic programming module to analyze for detecting the blood clots or vascular stenosis in time. Therefore, the present invention can avoid implanting urgently when the dialytic fistula is in poor internal condition in order to effectively save medical resources and improve medical quality. The present invention not only provides a device to monitor vascular conditions for dialysis patients by themselves, but also provides a diagnostic tool for clinicians to diagnose more accurately. Second, the present invention has several advantages such as portable design, immediate diagnosis, non-invasive characteristic, convenient use and so on. Third, the present invention has wireless-transmitting function for connecting to a medical network in order to effectively monitor and diagnose the internal condition of the dialytic fistula. Fourth, the present invention can be adapted to other electronic devices, such as PDA, mobile phone and so on for convenient use. Fifth, the present invention includes a storage device adapted to store and compare data.

Further benefits and advantages of the present invention will become apparent after a careful reading of the detailed description with appropriate reference to the accompanying drawings.

### BRIEF DESCRIPTION OF THE DRAWINGS

FIG. 1 is a perspective view of a portable dialysis access monitor device in accordance with the present invention;

FIG. 2 is a block diagram of the portable dialysis access monitor device in accordance with the present invention; and

FIG. 3 is a flowchart showing how to use the portable dialysis access monitor device in accordance with the present invention.

### DETAILED DESCRIPTION OF THE INVENTION

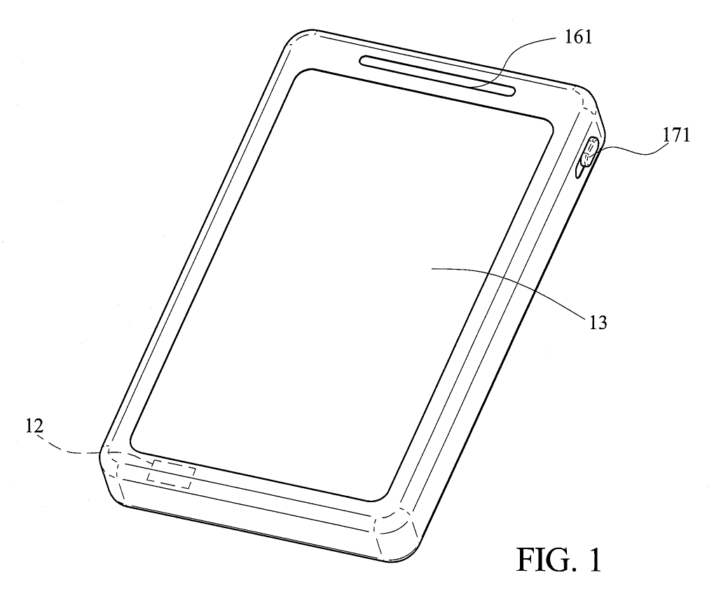

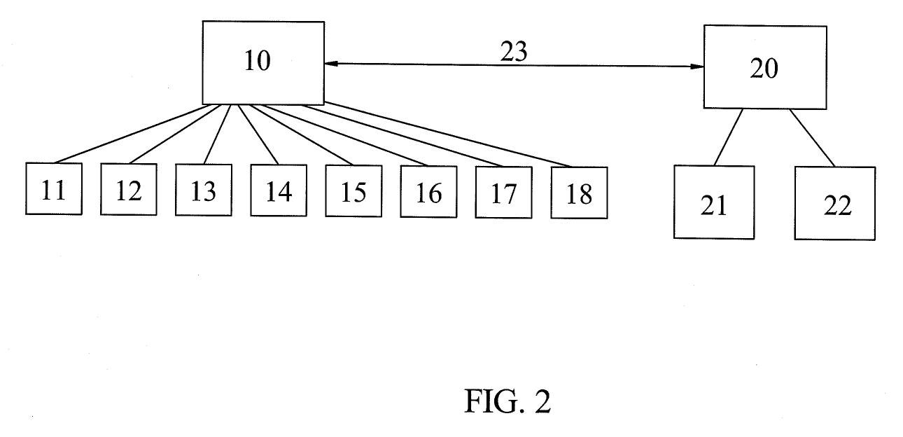

Referring to the drawings and initially to FIGS. 1-3, a portable dialysis access monitor device comprises an electronic monitor device 10 and a server 20. The electronic monitor device 10 includes a first microprocessor 11, an acoustic detector 12, a touch screen 13, a first storage device 14, a diagnostic programming module 15, an alarm device 16, a power supply device 17 and an interface 18 disposed between a human being and a machine. The first microprocessor 11 is electrically connected to the touch screen 13, the first storage device 14, the alarm device 16 and the power supply device 17 respectively. The interface 18 is a graphical user interface. The first microprocessor 11 collects acoustic frequency signals, sound signals and thrill signals detected from a dialytic fistula and stored in the first storage device 14. The first microprocessor 11 utilizes the diagnostic programming module 15 to diagnose. If signal difference is greater than a predetermined threshold value, the electronic monitor device 10 provides a warning via the alarm device 16.

Besides, the server 20 includes a second microprocessor 21, a second storage device 22 and a mobile internet 23. The server 20 is connected to the electronic monitor device 10 by utilizing the mobile internet 23. The server 20 receives and analyzes the acoustic frequency signals, the sound signals and the thrill (e.g., a vascular murmur) signals detected from the dialytic fistula by utilizing the second microprocessor 21 to get an analyzed result. The server 20 transmits the analyzed result to the electronic monitor device 10 by utilizing the mobile internet 23 and the analyzed result is stored in the second storage device 22. The present invention can combine several functions such as immediate diagnosing, medical service and etc. The server 20 can transmit the analyzed result and related information to attending physicians or hospitals cooperated with insurance companies.

Preferably, the electronic monitor device 10 is a PDA.

Preferably, the electronic monitor device 10 is a mobile phone.

Preferably, the alarm device 16 includes a speaker.

Preferably, the touch screen 13 is a liquid crystal display touch screen.

Preferably, the first storage device 14 is a memory card.

Preferably, the second storage device 22 is a hard disk.

Preferably, the power supply device 17 includes a lithium battery; the power supply device 17 further includes a power switch 171.

Further, in clinical observations, when vascular stenosis and vascular blockages happen, it can accompany changes of the blood thrill (attenuated or eliminated). It can also accompany changes of the bruit (e.g., an abnormal sound) including the changes of frequency and volume when utilizing a stethoscope in auscultation. In general, the partial occlusion of the blood flow causes the turbulence characteristics of the blood flow to change. These turbulence characteristics, in turn are manifested as vibrations or sounds that can be detected at the skin surface. As is well known, fluid flowing through a conduit produces turbulence that, in turn, may produce sounds in the audible frequency range. The characteristics of these audible turbulence sounds are determined by many variables, including conduit course and geometry, flow volume and velocity, conduit dimensions, conduit wall elasticity, etc. When the dialytic fistula is blocked, the lumen of the dialytic fistula becomes smaller to make the blood flow faster. More specifically, as fluid approaches a narrowed cross-section within the conduit the fluid velocity increases and the fluid flow characteristics in the narrowed region depend on the conduit geometry and flow properties.

The present invention provides a portable dialysis access monitor device. The device is adapted to calculate frequency and sound by utilizing a stethoscope and utilize a chip to convert analog data into digital data to proceed a quantitative analysis by programs. Hence, detecting possible vascular stenosis or vascular blockages can be provided for ensuring the reliability of the dialytic fistula. The present invention is adapted to cooperate with modern electronic devices such as mobile phone, PDA and so on. The present invention not only provides a device to monitor vascular conditions for dialysis patients by themselves but also provides a diagnostic tool for clinicians to diagnose more accurately. In the present invention, sensors can be mounted in any desired manner such as an artery end of the dialytic fistula, a vein end of the dialytic fistula and so on in order to facilitate the acquisition of vibrations and sounds associated with vascular blood flow turbulence. The present invention utilizes the first microprocessor 11 to process, digitalize the signals and compare the frequency and sound by software.

The comparison rule of the software is to acquire signals from a single cardiac cycle. Compare the signals acquired from different interval each other except the identical part. If signal difference is greater than a predetermined threshold value, the electronic monitor device 10 can provide a warning via the alarm device 16. A first analyzed result can be displayed on a screen. A second analyzed result can be obtained from the server 20. The second analyzed result can be digitalized to transmit to the electronic monitor device 10 via the mobile internet 23. Therefore, if the blood flow is abnormal, clinicians and dialysis patients can be notified to transfer to a hospital for further examination or invasive vascular treatment.

The calculation rule of the software is to utilize a wave analysis program to show the relation between waveform and time. Then, to acquire a single cardiac cycle for calculating and to obtain several values from the software including maximum wave amplitude (Amx; %), minimum wave amplitude (Amin, %), the time when the amplitude is greater than 50% maximum wave amplitude, the percentage of the time in a whole cardiac cycle, the proportion of spectrum area in a cardiac cycle and so on for further analyzing. By comparing and analyzing several major parameters at different interval such as maximum amplitude difference (Amxd; %), minimum amplitude difference (Amind; %), the time difference when the amplitude is greater than 50% maximum wave amplitude (T50Hd) and the percentage difference of the spectrum area. If these major parameters are greater than the predetermined values, the electronic monitor device 10 can provide a warning via the alarm device 16. Then, these major parameters are transmitted to the server 20 for further diagnosing and providing an intervening medical treatment.

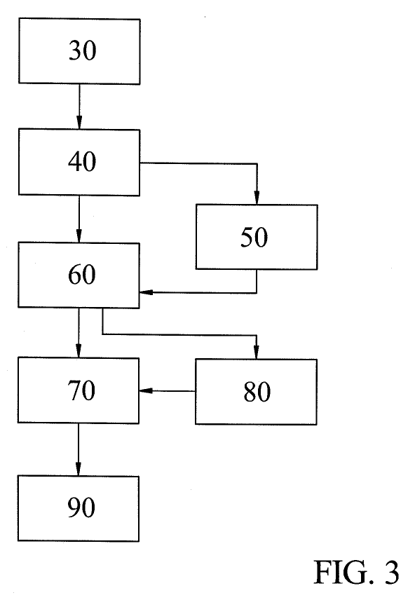

Additionally, referring to FIG. 3, it shows how to use the portable dialysis access monitor device of the invention. The present invention utilizes sensors to detect vibrations or sounds at or near the skin surface 30 and utilizes a first microprocessor to convert these vibrations or sounds into electrical signals 40 stored in the storage device 50. Further, the present invention utilizes a diagnostic programming module to compare and analyze the electrical signals 60 to get a diagnostic result displayed on the touch screen of the electronic monitor device 70. Still further, the diagnostic result is transmitted to the server 80. Therefore, a proper medical intervention 90 can be provided according to the diagnostic result.

By utilizing the invention, the following advantages are obtained. First, the present invention provides a portable dialysis access monitor device for detecting possible problems of the dialytic fistula and utilizes the diagnostic programming module 15 to analyze for detecting the blood clots or vascular stenosis in time. Therefore, the present invention can avoid implanting urgently when the dialytic fistula is in poor internal condition in order to effectively save medical resources and improve medical quality. The present invention not only provides a device to monitor vascular conditions for dialysis patients by themselves, but also provides a diagnostic tool for clinician to diagnose more accurately. Second, the present invention has several advantages such as portable design, immediate diagnosis, non-invasive characteristic, convenient use and so on. Third, the present invention has wireless-transmitting function for connecting to a medical network in order to effectively monitor and diagnose the internal condition of the dialytic fistula. Fourth, the present invention can be adapted to other electronic devices, such as Personal Digital Assistant (PDA), mobile phone and so on for convenient use. Fifth, the present invention includes the storage devices 14, 22 adapted to store and compare data.

Although the invention has been explained in relation to its preferred embodiment, it is to be understood that many other possible modifications and variations can be made without departing from the spirit and scope of the invention as hereinafter claimed.

1. A portable dialysis access monitor device, comprising:
an electronic monitor device comprising a first microprocessor, an acoustic detector, a touch screen, a first storage device, a diagnostic programming module, an alarm device, a power supply device, and an interface disposed between a human being and a machine wherein the first microprocessor is electrically connected to the touch screen, the first storage device, the alarm device, and the power supply device respectively; the interface is a graphical user interface; the first microprocessor collects acoustic frequency signals, sound signals and thrill signals detected from a dialytic fistula and stores in the first storage device; the first microprocessor utilizes the diagnostic programming module to diagnose; and if signal difference is greater than a predetermined threshold value, the electronic monitor device provides a warning via the alarm device; and
a server comprising a second microprocessor, a second storage device, and a mobile internet wherein the server is connected to the electronic monitor device via the mobile internet; the server receives and analyzes the acoustic frequency signals, the sound signals and the thrill signals detected from the dialytic fistula by utilizing the second microprocessor to get an analyzed result; the server transmits the analyzed result to the electronic monitor device via the mobile internet; and the analyzed result is stored in the second storage device.

2. The device of claim 1, wherein the electronic monitor device is a PDA.

3. The device of claim 1, wherein the electronic monitor device is a mobile phone.

4. The device of claim 1, wherein the alarm device includes a speaker.

5. The device of claim 1, wherein the touch screen is a liquid crystal display touch screen.

6. The device of claim 1, wherein the first storage device is a memory card.

7. The device of claim 1, wherein the second storage device is a hard disk.

8. The device of claim 1, wherein the power supply device includes a lithium battery and a power switch.
